(19) United States
(12) Patent Application Publication
(10) Pub. No.: US 2007/0059265 A1
(43) Pub. Date: Mar. 15, 2007

(54) Benzylidene-1,3-thiazolidine-2,4-dione compounds for stimulating or inducing the growth and/or for reducing the loss and/or for increasing the density of keratin fibers

(75) Inventor: Christophe Boulle, Lagny S/Marne (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/476,123

(22) Filed: Jun. 28, 2006

Related U.S. Application Data
(60) Provisional application No. 60/697,972, filed on Jul. 12, 2005.

(30) Foreign Application Priority Data
Jun. 28, 2005 (FR) 05/51793

(51) Int. Cl. A61K 31/426; A61K 8/49

(52) U.S. Cl. 424/70.1; 514/369

(57) ABSTRACT

The benzylidene-1,3-thiazolidine-2,4-dione compounds having the structural formula (I), or salt and/or solvate and/or isomer thereof: 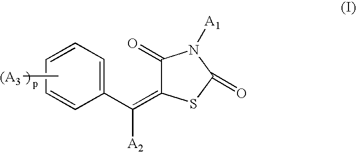 are useful active agents for inducing and/or stimulating the growth of mammalian keratin fibers and/or reducing the loss and/or increasing the density thereof, particularly of human head hair, beard hair, moustache hair, eyelashes and eyebrows, and advantageously are active agents for caring for or making up the hair or the eyelashes.

### CROSS-REFERENCE TO PRIORITY/PROVISIONAL APPLICATIONS

This application claims priority under 35 U.S.C. § 119 of FR 05/51793, filed Jun. 28, 2005, and of U.S. Provisional Application No. 60/697,972, filed Jul. 12, 2005, each hereby expressly incorporated by reference and each assigned to the assignee hereof.

### CROSS-REFERENCE TO COMPANION APPLICATIONS

Copending applications Ser. No. ______ [Attorney Docket No. 1016800-000769], Ser. No. ______ [Attorney Docket No. 1016800-000770], and Ser. No. ______ [Attorney Docket No. 1016800-000771], filed concurrently herewith, each hereby also expressly incorporated by reference and each also assigned to the assignee hereof.

### BACKGROUND OF THE INVENTION

1. Technical Field of the Invention

The present invention relates to the administration of an effective amount of at least one benzylidene-1,3-thiazolidine-2,4-dione compound of particular structure as an active agent for inducing and/or stimulating the growth of keratin fibers, especially human keratin fibers, and/or for reducing the loss and/or increasing the density thereof.

More especially, this invention relates to compositions for caring for or making up the hair or the eyelashes, to induce and/or to stimulate the growth and/or to reduce the loss and/or to increase the density thereof.

The present invention also relates to novel benzylidene-1,3-thiazolidine-2,4-dione compounds and to a cosmetic/therapeutic regime or regimen for stimulating the growth and/or reducing the loss of keratin fibers and in particular of the hair.

The human keratin fibers to which this invention relates are especially head hair, the eyebrows, the eyelashes, pubic hair, beard hair and moustache hair. The invention more especially relates to human head hair and/or eyelashes.

In particular, the present invention relates to compositions for topical application for caring for and/or making up the hair or the eyelashes, containing at least one benzylidene-1,3-thiazolidine-2,4-dione compound of particular formula, to increase their density and/or to improve their appearance.

2. Description of Background and/or Related and/or Prior Art

Hair growth and hair renewal are mainly determined by the activity of the hair follicles and of their matrix environment. Their activity is cyclical and comprises essentially three phases, namely, the anagenic phase, the catagenic phase and the telogenic phase.

The anagenic phase (active phase or growth phase), which lasts several years and during which the hair gets longer, is followed by a very short and transient catagenic phase that lasts a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ends up by falling out. At the end of this rest period, a new follicle is regenerated in situ and another cycle begins.

The head of hair is thus under permanent renewal, and, out of the approximately 150,000 hairs that make up a head of hair, about 10% are at rest and will be replaced within a few months.

The natural loss or falling-out of the hair may be estimated, on average, as being a few hundred hairs per day for a normal physiological state. This process of permanent physical renewal undergoes a natural change during aging, the hairs become finer and their cycles shorter.

In addition, various causes may result in a substantial, temporary or permanent loss of hair. This may be loss and impairment of hair at the terminal stage of pregnancy (post-partum), during states of dietary malnutrition or imbalance, during physiological stress, or during states of asthenia or of hormonal dysfunction, as may be the case during or at the terminal stage of the menopause. It may also be a case of loss or impairment of the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. The successive growth cycles result in hairs that are finer and finer and shorter and shorter, gradually transforming into an unpigmented down. Certain areas are preferentially affected, especially the temporal or frontal lobes in men, and a diffuse alopecia of the crown of the head is observed in women.

The term alopecia also comprehends a whole family of afflictions of hair follicles whose final consequence is the permanent, partial or general loss of the hair. This is more particularly a matter of androgenic alopecia. In a large number of cases, early loss of hair occurs in genetically predisposed individuals; this is then a matter of andro-chrono-genetic alopecia. This form of alopecia especially affects men.

In certain dermatoses of the scalp with an inflammatory component, for instance psoriasis or seborrhoeic dermatitis, hair loss may be greatly accentuated or may result in highly disrupted follicular cycles.

The cosmetics and pharmaceutical industries have for many years been investigating compositions for eliminating or reducing alopecia, and especially for inducing or stimulating hair growth or reducing its loss.

In this perspective, a large number of compositions comprising very diverse active agents have already been proposed, for instance 2,4-diamino-6-piperidinopyrimidine 3-oxide, or “minoxidil”, described in U.S. Pat. Nos. 4,139,619 and 4,596,812, or the numerous derivatives thereof such as those described in EP-0-353,123, EP-0-356,271, EP-0-408,442, EP-0-522,964, EP-0-420,707, EP-0-459,890 and EP-0-519,819.

Clinical studies have shown that PGF2-α analogues have the property of inducing the growth of body hairs and eyelashes in man and animals (Murray A. and Johnstone M. D., 1997, Am. J. Opht., 124(4), 544-547). In man, tests performed on the scalp have shown that a prostaglandin E2 analogue (viprostol) has the property of increasing the hair density (Roenigk H. H., 1988, Clinic Dermatol., 6(4), 119-121).

Moreover, WO 98/33497 describes pharmaceutical compositions containing prostaglandins or prostaglandin derivatives, for combating hair loss in man. Prostaglandins of the type A2, F2α and E2 are mentioned as being preferred.

However, prostaglandins are molecules with a very short biological half-life, which act in an autocrine or paracrine manner, this reflecting the local and labile nature of the metabolism of prostaglandins (Narumiya S. et al., 1999, Physiol. Rev., 79(4), 1193-1226).

It is thus seen to be important, in order to maintain and/or increase the hair density in man, to preserve the endogenous reserves of PGF2-α and similarly of PGE2 in various compartments of the hair follicle or its immediate cutaneous environment.

One solution that gives good results is the use of lipoxygenase-inhibiting compounds and/or cyclooxygenase-inducing compounds to promote hair growth; one theory is that the use of such compounds directs the metabolism of fatty acids towards the endogenous synthesis of prostaglandins in preference to other routes.

However, to further improve the results, it would be desirable to be able to prolong the activity of the prostaglandins involved in growing the hair and keeping it alive.

It is moreover well known that differentiation of the keratinocytes of the epidermis and of the hair follicle are clearly different. Thus, it is known that the keratins of the hair shaft represent a family (Langbein et al., 2001, J. Biol. Chem., 276: 35123-35132) that is different from the one expressed in the epidermis, that differentiation markers such as the keratins K₁ and K₁₀ are not expressed in the hair follicle and in particular in the outer sheath (Lenoir et al., 1988, Dev. Biol., 130: 610-620), that trichohyalin (O'Guin et al., 1992, J. Invest. Dermatol., 98: 24-32) and keratin K6irs (Porter et al., 2001, Br. J. Dermatol., 145: 558-568) are expressed in the hair follicle, in particular in the inner sheath, but not in the epidermis, and that type-1 cyclooxygenase, although expressed in the epidermis, is not expressed in the keratinocytes of the hair follicle but in the dermal papilla (Michelet. et al., 1997, J. Invest. Dermatol., 108: 205-209).

### SUMMARY OF THE INVENTION

It has now been demonstrated that an enzyme specifically involved in the degradation of these prostaglandins is present in the dermal papilla of the hair, which is a compartment that is a decisive factor in the life of the hair. Specifically, the presence of type-1 15-hydroxyprostaglandin dehydrogenase (abbreviated to 15-PGDH) at this level has now been proven. It has also now been shown that the inhibition of type-1 15-PGDH has a beneficial effect on hair growth.

Thus, the present invention features a haircare or hair treatment composition containing at least one particular inhibitor of type-1 15-hydroxyprostaglandin dehydrogenase and a physiologically acceptable medium.

Type-1 15-PGDH is a key enzyme in the deactivation of prostaglandins, in particular of PGF2-α and PGE2, which are important mediators of hair growth and survival. It corresponds to the classification EC 1.1.1.141 and is NAD⁺-dependent. It has been isolated from pig kidney; its inhibition with a thyroid hormone, triiodothyronine, at doses very much higher than the physiological doses, has especially been observed. Type-2 15-PGDH is itself NADP-dependent.

It has now been found that certain benzylidene-1,3-thiazolidine-2,4-dione compounds of formula (I) that will be defined in detail later and/or salts thereof and/or mineral solvates thereof and/or cis-trans or Z/E isomers thereof are, surprisingly, endowed with favorable activity on improving the density of human keratin fibers. It has, moreover, also been found that these compounds are inhibitors of type-1 15-hydroxyprostaglandin dehydrogenase.

The present invention thus also features the administration of an effective amount of at least one benzylidene-1,3-thiazolidine-2,4-dione compound of formula (I) below, or a salt and/or solvate and/or cis-trans or Z/E isomer thereof: 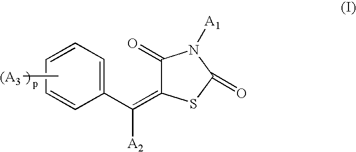
in which:

a) A₁ and A₂ simultaneously are hydrogen;

b) A₃ is a group Z₁;

c) Z₁ is a group OZ₂ in which Z₂ is a phenyl group optionally substituted with one or more adamantyl, CF₃, halogen or linear or branched, saturated or unsaturated C₁-C₂₀ alkyl radicals; and

d) p ranges from 0 to 5 inclusive and, when p>1, the substituents A₃ may be identical or different; as an active agent for inducing and/or stimulating the growth of keratin fibers, especially human keratin fibers, and/or for reducing the loss and/or increasing the density thereof.

### DETAILED DESCRIPTION OF BEST MODE AND SPECIFIC/PREFERRED EMBODIMENTS OF THE INVENTION

According to the invention, the term “salts of the compound of formula (I)” means the organic or mineral salts of a compound of formula (I).

As mineral salts according to the invention, exemplary are the sodium or potassium salts and also the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts; hydroxides, hydrohalides (for example hydrochlorides), carbonates, hydrogen carbonates, sulfates, hydrogen phosphates, phosphates.

The organic salts according to the invention are, for example, the triethanolamine, monoethanolamine, ethanolamine, hexadecylamine and N,N,N′,N′-tetrakis(2-hydroxypropyl)ethylenediamine salts, and also organic acid salts, for instance citrates, lactates, glycolates, gluconates, acetates, propionates, fumarates, oxalates and tartrates.

As possible solvates of the compounds of formula (I), exemplary are the hydrates, alcoholates and hydroalcoholates.

According to the invention, the compounds of formula (I) are in isolated form, i.e., non-polymeric form.

According to the invention, the term “at least one” means one or more (2, 3 or more). In particular, the composition may contain one or more compounds of formula (I). This or these compound(s) may be cis-trans or Z/E isomers or a mixture of cis-trans or Z/E isomers. They may also be in tautomeric form. This or these compound(s) may be enantiomers and/or diastereoisomers or a mixture of these isomers, in particular a racemic mixture.

For the purposes of the invention, the term “alkyl radical” means a hydrocarbon-based radical that may be linear or branched and saturated or unsaturated. In particular, the alkyl radical contains from 1 to 20 and preferably from 1 to 10 carbon atoms. As examples of alkyl radicals that may be used in the invention, representative are methyl, ethyl, isopropyl, n-butyl, tert-butyl, n-hexyl, 2-ethylhexyl, ethylene and propylene radicals.

As halogen atoms according to the invention, exemplary are chlorine, fluorine or bromine atoms, and better still chlorine and fluorine atoms.

Among the compounds of formula (I) in accordance with the invention that are more particularly exemplary are those selected from the following list of compounds:

| Com- | |
|---|---|
| pound | Structure |
| 1 | 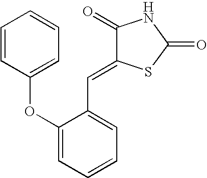 |
| 2 | 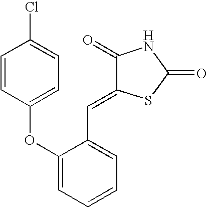 |
| 3 | 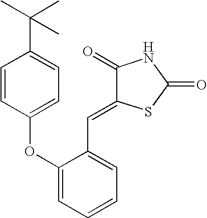 |
| 4 | 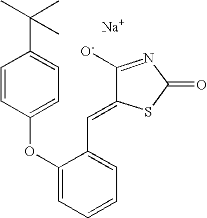 |
| 5 | 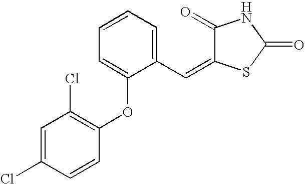 |
| 6 | 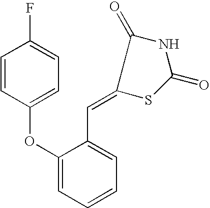 |
| 7 | 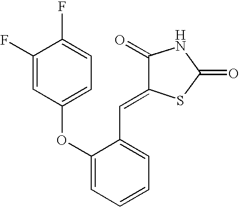 |
| 8 | 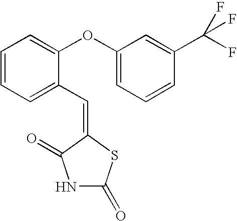 |
| 9 | 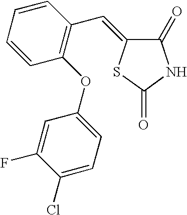 |
| 10 | 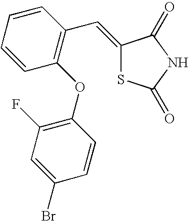 |
| 11 | 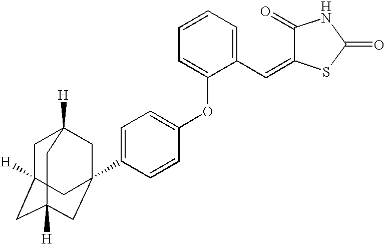 |
| 12 | 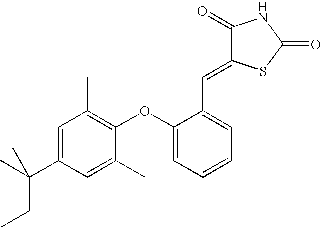 |
| 13 | 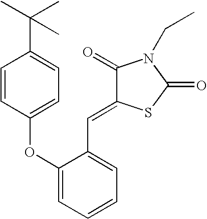 |
| 14 | 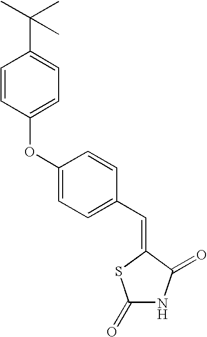 |
| 15 | 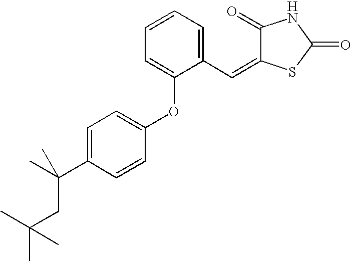 |
| 16 | 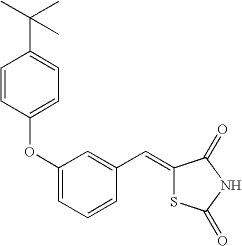 |
| 17 | 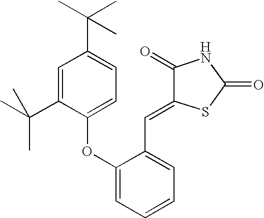 |

The compounds that are more particularly preferred are selected from among:

The compound (5Z)-5-[2-(3,4-difluorophenoxy)benzylidene]-1,3-thiazolidine-2,4-dione of structure:

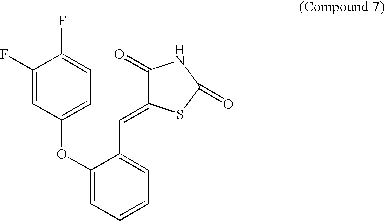

The compound (5Z)-5-[2-(4-tert-butylphenoxy)benzylidene]-1,3-thiazolidine-2,4-dione of structure:

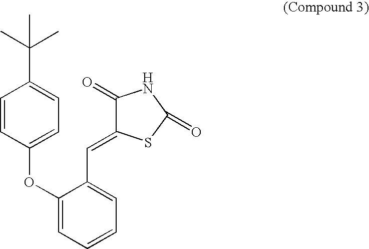

Its sodium salt of structure:

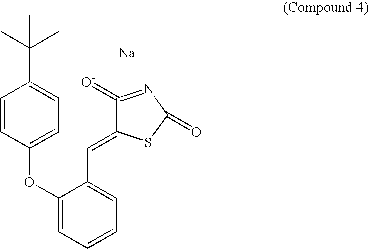

The compound (5Z)-5-[2-(4-chloro-3-fluorophenoxy)benzylidene]-1,3-thiazolidine-2,4-dione of structure:

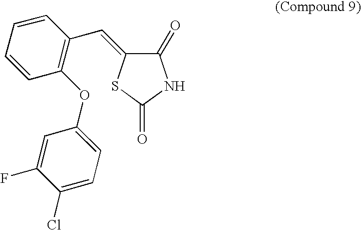

The compound (5Z)-5-[4-(4-tert-butylphenoxy)benzylidene]-1,3-thiazolidine-2,4-dione of structure: 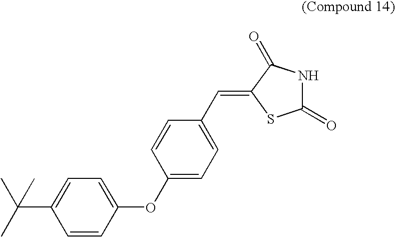
and also the salts and/or solvates and/or cis-trans or Z/E isomers thereof.

The compounds of formula (I) in accordance with the invention may be obtained according to a synthetic route as described below in the reaction scheme defined below. The synthetic route used entails an aromatic nucleophilic substitution, then a Knoevenagel condensation.

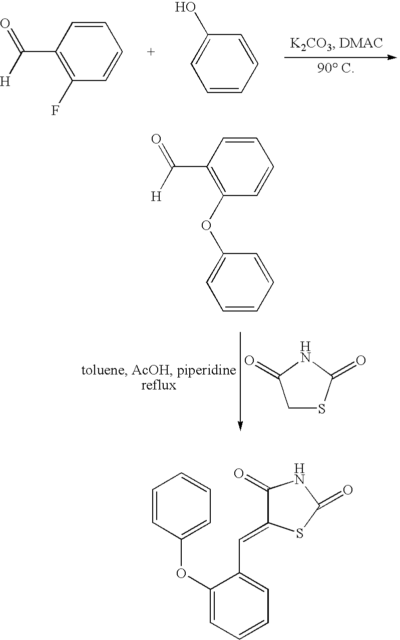

Certain of the compounds of formula (I) are known per se. Other compounds are novel and constitute another aspect of the invention.

One family of novel compounds in accordance with the invention includes those corresponding to formula (Ia): 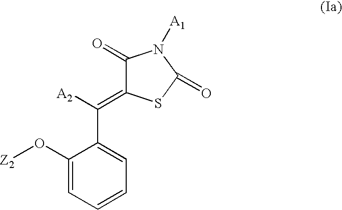
in which

A₁ and A₂ are each hydrogen;

Z₂ is a phenyl group substituted with one or more adamantyl or linear or branched, saturated or unsaturated C₂-C₂₀ alkyl radicals, with the proviso that said alkyl radicals are other than tert-butyl and isobutyl.

Examples of compounds of formula (Ia) that are representative include the following compounds:

The compound (5Z)-5-[2-[4-(1-adamantylphenoxy)benzylidene]-1,3-thiazolidine-2,4-dione (Compound 11) of structure:

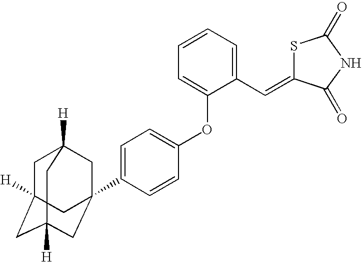

The compound (5Z)-5-[2-(2,6-dimethyl-4-tert-pentylphenoxy)benzylidene]-1,3-thiazolidine-2,4-dione (Compound 12) of structure:

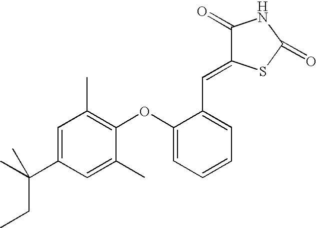

Compound (15) of structure: 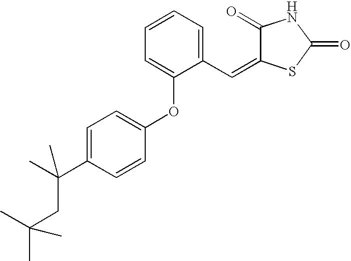
and also the salts and/or solvates and/or cis-trans or Z/E isomers thereof.

The present invention also features the administration, especially the cosmetic administration, of at least one benzylidene-1,3-thiazolidine-2,4-dione compound of formula (I) or a salt and/or solvate and/or cis-trans or Z/E isomer thereof, as defined above, as an active agent for inducing and/or stimulating the growth of keratin fibers, in particular human keratin fibers, and/or for reducing their loss and/or increasing their density.

This invention also features the formulation of at least one benzylidene-1,3-thiazolidine-2,4-dione compound of formula (I) or a salt and/or solvate and/or cis-trans or Z/E isomer thereof, into cosmetic compositions for caring for or treating keratin fibers, especially human keratin fibers, to reduce their loss and/or increase their density. These compositions also make it possible to keep the keratin fibers in good condition and/or to improve their appearance.

The present invention also features the use of at least one benzylidene-1,3-thiazolidine-2,4-dione compound of formula (I) or a salt and/or solvate and/or cis-trans or Z/E isomer thereof, for the manufacture of compositions for caring for or treating keratin fibers, especially human keratin fibers, to induce and/or stimulate the growth of the said fibers and/or to reduce their loss and/or increase their density.

This invention also applies to the keratin fibers of mammalian animals (for example dogs, horses or cats).

The human keratin fibers to which the invention applies are especially head hair, the eyebrows, the eyelashes, beard hair and moustache hair.

Thus, the present invention also features the formulation of at least one benzylidene-1,3-thiazolidine-2,4-dione compound of formula (I) or a salt and/or solvate and/or cis-trans or Z/E isomer thereof, into human cosmetic haircare compositions for reducing the loss and/or increasing the density of the hair and/or for treating andro-chrono-genetic alopecia.

This invention also features the use of at least one benzylidene-1,3-thiazolidine-2,4-dione compound of formula (I) or a salt and/or solvate and/or cis-trans or Z/E isomer thereof, for the preparation of human hair compositions for inducing and/or stimulating the growth and/or reducing the loss and/or increasing the density of the hair and/or for treating androgenic alopecia.

The present invention also features the formulation of at least one benzylidene-1,3-thiazolidine-2,4-dione compound of formula (I) or a salt and/or solvate and/or cis-trans or Z/E isomer thereof, into human cosmetic haircare compositions for treating alopecia of natural origin and in particular androgenic alopecia.

This invention also features the use of at least one benzylidene-1,3-thiazolidine-2,4-dione compound of formula (I) or a salt and/or solvate and/or cis-trans or Z/E isomer thereof, for the preparation of human haircare compositions for treating alopecia of natural origin and in particular androgenic alopecia. Thus, these compositions make it possible to keep the head of hair in good condition and/or to combat natural hair loss, more especially in men.

This invention also features the formulation of at least one benzylidene-1,3-thiazolidine-2,4-dione compound or a salt and/or solvate and/or cis-trans or Z/E isomer thereof, into cosmetic compositions for caring for and/or making up human eyelashes, to induce and/or stimulate the growth and/or increase the density of the eyelashes.

The present invention also features the use of at least one benzylidene-1,3-thiazolidine-2,4-dione compound of formula (I) or a salt and/or solvate and/or cis-trans or Z/E isomer thereof, for the preparation of compositions for caring for or treating human eyelashes, to induce and/or stimulate the growth and/or to increase the density of the eyelashes. These compositions thus make it possible to keep the eyelashes in good condition and/or to improve their condition and/or appearance.

The present invention also features the administration of at least one benzylidene-1,3-thiazolidine-2,4-dione compound of formula (I) or a salt and/or solvate and/or cis-trans or Z/E isomer thereof, as an inhibitor of type-1 15-hydroxyprostaglandin dehydrogenase especially of human skin.

This invention also features the use of at least one benzylidene-1,3-thiazolidine-2,4-dione compound of formula (I) or a salt and/or solvate and/or cis-trans or Z/E isomer thereof, for the manufacture of a composition for treating disorders associated with the presence of type-1 15-hydroxyprostaglandin dehydrogenase, in particular in man.

The present invention also features a cosmetic/therapeutic regime or regimen for treating human keratin fibers (especially the hair or the eyelashes) and/or the skin from which the said fibers emerge, including the scalp and the eyelids, comprising topically applying onto the said keratin fibers and/or the said skin a cosmetic composition which comprises at least one benzylidene-1,3-thiazolidine-2,4-dione compound of formula (I) or a salt and/or solvate and/or cis-trans or Z/E isomer thereof, leaving it in contact with the keratin fibers and/or the skin from which the said fibers emerge, and optionally rinsing the said fibers and/or the said skin.

This treatment regime or regimen does indeed have the characteristics of a cosmetic process in so far as it makes it possible to improve the aesthetic appearance of human keratin fibers and especially the hair and the eyelashes by giving them greater vigor and an improved appearance. In addition, it may be carried out daily for several months, without medical prescription.

More especially, the present invention features a cosmetic regime or regimen for caring for human hair and/or the scalp, to improve their condition and/or appearance, comprising topically applying to the hair and/or the scalp a cosmetic composition which comprises an effective amount of at least one benzylidene-1,3-thiazolidine-2,4-dione compound of formula (I) or a salt and/or solvate and/or cis-trans or Z/E isomer thereof, leaving it in contact with the hair and/or the scalp, and optionally rinsing the hair and/or the scalp.

The present invention also features a cosmetic regime or regimen for caring for and/or making up human eyelashes, to improve their condition and/or appearance, comprising topically applying to the eyelashes and/or the eyelids a mascara composition which comprises at least one benzylidene-1,3-thiazolidine-2,4-dione compound of formula (I) or a salt and/or solvate and/or cis-trans or Z/E isomer thereof, and leaving it in contact with the eyelashes and/or the eyelids. This mascara composition may be applied alone or as a base coat for a standard pigmented mascara and may be removed like a standard pigmented mascara.

The present invention also features care and/or mascara compositions for the hair or the eyelashes, containing a cosmetically acceptable medium for topical application and a benzylidene-1,3-thiazolidine-2,4-dione compound of formula (I) or a salt and/or solvate and/or cis-trans or Z/E isomer thereof.

This invention also features compositions for caring for and/or making up keratin fibers, comprising, in a physiologically acceptable medium, in particular a cosmetic medium, at least one benzylidene-1,3-thiazolidine-2,4-dione compound or a salt and/or solvate and/or cis-trans or Z/E isomer thereof and at least one additional active agent that promotes the regrowth and/or limits the loss of human keratin fibers, selected from among aminexil, FP receptor agonists, prostaglandins and derivatives thereof, and vasodilators and more especially selected from among aminexil, minoxidil, latanoprost, (5E)-7-{(1R,2R,3R,5S)-3,5-dihydroxy-2-[(3R)-3-hydroxy-5-phenylpentyl]cyclopentyl}hept-5-enoic acid, butaprost and travoprost.

This invention also features the cosmetic administration of a benzylidene-1,3-thiazolidine-2,4-dione compound of formula (I) or a salt and/or solvate and/or cis-trans or Z/E isomer thereof, as an active agent for preserving the amount and/or the activity of the prostaglandins in the hair follicles.

This invention also features the use of at least one benzylidene-1,3-thiazolidine-2,4-dione compound of formula (I) or a salt and/or solvate and/or cis-trans or Z/E isomer thereof, for the manufacture of a composition for preserving the amount and/or the activity of the prostaglandins in the hair follicles.

The term “15-hydroxyprostaglandin dehydrogenase inhibitor” means a compound of formula (I) capable of inhibiting or reducing the activity of the enzyme type-1 15-PGDH, in particular human type-1 15-PGDH, and/or capable of inhibiting, reducing or slowing down the reaction catalyzed by this enzyme.

According to one advantageous embodiment of the invention, the compound of formula (I) is a specific inhibitor of 15-PGDH; the term “specific inhibitor” means an active agent that has little or no inhibitory effect on prostaglandin synthesis, in particular on the synthesis of PGF2-α or PGE2. According to one particular embodiment of the invention, the inhibitor of 15-PGDH has little or no inhibitory effect on prostaglandin synthesis, in particular on the synthesis of PGF2-α or PGE2. In particular, the inhibitor of type-1 15-PGDH has little or no inhibitory effect on prostaglandin synthase (abbreviated to PGF synthase or PGFS).

Specifically, it has now been found that PGF synthase is also expressed in the dermal papilla. Maintenance of an effective amount of prostaglandins at the site of action thus results from a complex biological equilibrium from the synthesis and the degradation of these molecules. The exogenous supply of compounds that inhibit catabolism will thus be less effective if this activity is combined with an inhibition of the synthesis of these prostaglandins.

Advantageously, the compounds of formula (I), in salified or non-salified, solvated or non-solvated form, have inhibitory activity on 15-PGDH that is higher than the inhibitory activity on PGF synthase. These compound, are called selective inhibitors of

15-PGDH with respect to PGF synthase. In particular, the ratio from the inhibitory activities on PGF synthase and on 15-PGDH, respectively, for a given concentration, determined especially by the concentration that inhibits 50% of the enzymatic activity of PGF synthase (IC_{50fs}) relative to the concentration that inhibits 50% of the enzymatic activity of 15-PGDH (IC_{50dh}) is at least greater than 1 and especially at least 3:1 and advantageously greater than or equal to 5:1.

In the text hereinbelow, and unless otherwise indicated, the use of the term “compound of formula (I)” should be understood as meaning either the compound of formula (I) or a salt or solvate and in particular a hydrate thereof, or a solvated salt (in particular a hydrated salt) thereof and cis-trans or Z/E isomers.

The effective amount of a compound of formula (I) (salified or non-salified, solvated or non-solvated) corresponds to the amount necessary to obtain the desired result (namely to increase the density or promote the growth of keratin fibers and in particular of the hair or the eyelashes). One skilled in this art is therefore capable of evaluating this effective amount, which depends on the nature of the compound used, on the individual to whom it is applied and on the length of time of this application.

In the text hereinbelow, and unless otherwise indicated, the amounts of the various ingredients in the composition are given as weight percentages relative to the total weight of the composition.

To provide an order of magnitude, according to the invention, the compound of formula (I) (salified or non-salified, and solvated or non-solvated) or a mixture of compounds of formula (I) and/or salts thereof may be used in an amount ranging from 10⁻³% to 10% of the total weight of the composition, preferably in an amount representing from 10⁻³% to 5% and better still from 10⁻²% to 2% of the total weight of the composition, for example from 0.5% to 2%.

The compositions of the invention may be for cosmetic or pharmaceutical application. The compositions of the invention are preferably for cosmetic application. In addition, the composition must contain a non-toxic, physiologically acceptable medium that can be applied to human skin, including the scalp and the eyelids, and to human keratin fibers. For the purposes of the invention, the term “cosmetic” means a composition of pleasant appearance, odor and feel.

The compounds of formula (I), salified or non-salified, and solvated or non-solvated, may be formulated into compositions that should be ingested, injected or applied to the skin or to keratin fibers (to any area of skin or fibers to be treated).

According to the invention, the compound of formula (I) or a mixture of compounds of formula (I) may be used orally in an amount of from 0.1 to 300 mg per day, for example from 5 to 10 mg/day.

A preferred composition of the invention is a composition for cosmetic use and in particular for topical application to the skin and keratin fibers, and more especially to the scalp, the hair and the eyelashes.

These compositions may be in any known presentation form that is suitable for the mode of use.

For topical application to the skin or keratin fibers, the composition may be in the form of an aqueous, alcoholic or aqueous-alcoholic solution or suspension, or an oily suspension or solution, an emulsion or dispersion of more or less fluid consistency and especially of liquid or semi-liquid consistency, obtained by dispersion of a fatty phase in an aqueous phase (O/W) or conversely (W/O), a solid (O/W) or (W/O) emulsion or dispersion, a more or less fluid or solid aqueous, aqueous-alcoholic or oily gel, a free or compacted powder to be used in unmodified form or to be incorporated into a physiologically acceptable medium, or, alternatively, microcapsules, microparticles or vesicular dispersions of ionic and/or nonionic type.

A composition in the form of a foam or, alternatively, in the form of a spray or aerosol, then comprising a pressurized propellant, may also be administered.

It may thus be in the form of a lotion, serum, milk, O/W or W/O cream, gel, unguent, ointment, powder, balm, patch, impregnated pad, cake or foam.

In particular, the composition for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the compositions to which the invention applies may be in the form of a pigmented or unpigmented mascara, to be applied with a brush to the eyelashes or, alternatively, to beard or moustache hair.

For a composition for use by injection, the composition may be in the form of an aqueous lotion or an oily suspension. For oral use, the composition may be in the form of capsules, granules, drinkable syrups or tablets.

According to one particular embodiment, the composition according to the invention is in the form of a hair cream or hair lotion, a shampoo, a hair conditioner, a hair mascara or an eyelash mascara.

The amounts of the various constituents of the composition according to the invention are those generally employed in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase is from 5% to 99.9% by weight.

The fatty phase may contain fatty or oily compounds that are liquid at room temperature (25° C.) and atmospheric pressure (760 mm Hg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or, alternatively, acetone or ether.

The emulsifiers and co-emulsifiers used to obtain a composition in emulsion form are those generally employed in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5% to 20% by weight and better still from 1% to 8% by weight. The emulsion may also contain lipid vesicles and especially liposomes.

When the composition is in the form of an oily solution or gel, the fatty phase may represent more than 90% of the total weight of the composition.

Advantageously, for a topical hair application, the composition is an aqueous, alcoholic or aqueous-alcoholic solution or suspension and better still a water/ethanol solution or suspension. The alcoholic fraction may represent from 5% to 99.9% and better still from 8% to 80%.

For a topical mascara application, the composition of the invention is especially in the form of a wax-in-water or wax-in-oil dispersion, a gelled oil or an aqueous gel, which may be pigmented or unpigmented.

The compositions of the invention may also comprise other additional ingredients usually employed in the fields under consideration, selected from among solvents, aqueous-phase or oily-phase thickeners or gelling agents, dyestuffs that are soluble in the medium of the composition, solid particles such as fillers or pigments, antioxidants, preservatives, fragrances, electrolytes, neutralizers, film-forming polymers, UV blockers, for instance sunscreens, cosmetic and pharmaceutical active agents with a beneficial effect on the skin or keratin fibers, other than the compounds of formula (I), and mixtures thereof. These additives may be present in the composition in the amounts generally employed in cosmetics and dermatology, and especially in a proportion of from 0.01% to 50% and better still from 0.1% to 20%, for example from 0.1% to 10%, relative to the total weight of the composition. Depending on their nature, these additives may be introduced into the fatty phase, into the aqueous phase and/or into the lipid vesicles and especially liposomes.

Needless to say, one skilled in this art will take care to select the optional additional ingredients and/or the amount thereof such that the advantageous properties of the compositions according to the invention, i.e., the inhibition of type-1 15-PGDH and in particular the increase in the density of keratin fibers are not, or are not substantially, adversely affected by the envisaged addition.

As solvents according to the invention, exemplary are C₂ to C₈ lower alcohols, for instance ethanol, isopropanol, propylene glycol and certain light cosmetic oils, for instance C₆ to C₁₆ alkanes. Mention may also be made of benzyl alcohol.

As oils according to the invention, exemplary are oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that are exemplary include silicone waxes, beeswax, rice wax, candelilla wax, carnauba wax, paraffin wax and polyethylene wax.

As emulsifiers according to the invention, examples include glyceryl stearate, glyceryl laurate, sorbitol stearates, sorbitol oleates, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion.

As hydrophilic gelling agents according to the invention, exemplary are carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, exemplary are modified clays, for instance Bentones, metal salts of fatty acids, for instance aluminum stearates, hydrophobic-treated silica and ethylcellulose, and mixtures thereof.

As cosmetic or pharmaceutical active agents other than the compounds of formula (I), which may be used in the invention, exemplary are hydrophilic active agents such as proteins or protein hydrolysates, amino acids, polyols, urea, allantoin, sugars and sugar derivatives, water-soluble vitamins, plant extracts (those from Iridacea plants or from soybean) and hydroxy acids such as fruit acids or salicylic acid; and lipophilic active agents such as retinol (vitamin A) and its derivatives, especially an ester (retinyl palmitate), tocopherol (vitamin E) and its derivatives, especially an ester (tocopheryl acetate), essential fatty acids, ceramides, essential oils, salicylic acid derivatives, for instance 5octanoylsalicylic acid, hydroxy acid esters, and phospholipids, for instance lecithin, and mixtures thereof.

According to one particular embodiment of the invention, the compound of formula (I) or a salt and/or solvate thereof may be combined with additional compounds that promote the regrowth and/or limit the loss of keratin fibers (hair or eyelashes). These additional compounds are selected especially from among the lipoxygenase inhibitors as described in EP-648,488, the bradykinin inhibitors described especially in EP-845,700, prostaglandins and derivatives thereof, especially those described in WO 98/33497, WO 95/11003, JP 97-100091 and JP 96-134242, prostaglandin receptor agonists or antagonists, the non-prostanoic prostaglandin analogues as described in EP-1-175,891, EP-1-175,890, WO 01/74307, WO 01/74313, WO 01/74314, WO 01/74315 or WO 01/72268, and mixtures thereof.

As other additional active compounds that promote the growth of keratin fibers and/or limit their loss (particularly the hair and the eyelashes), which may be present in the compositions according to the invention, exemplary are vasodilators, anti-androgens, cyclosporins and analogues thereof, antimicrobial and antifungal agents, anti-inflammatory agents, and retinoids, alone or in a mixture.

The vasodilators are especially potassium-channel agonists, including minoxidil, and also the compounds described in U.S. Pat. Nos. 3,382,247, 5,756,092, 5,772,990, 5,760,043, 5,466,694, 5,438,058 and 4,973,474, cromakalim, nicorandil and diaxozide, alone or in combination.

The anti-androgens that may be used especially include steroidal or non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in U.S. Pat. Nos. 5,411,981, 5,565,467 and 4,910,226.

The antimicrobial or antifungal compounds may be selected from among selenium derivatives, octopirox, triclocarban, triclosan, zinc pyrithione, itraconazole, asiatic acid, hinokitiol, mipirocine, tetracyclines, especially erythromycin and the compounds described in EP-0-680,745, clinycin hydrochloride, benzoyl peroxide or benzyl peroxide, minocycline and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl nicotinate or hexyl nicotinate.

The anti-inflammatory agents may be selected from among steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine, salicylic acid and the compounds described in EP-0-770,399, WO 94/06434 and FR-2-268,523.

The retinoids may be selected from among isotretinoin, acitretin, tazarotene, retinal and adapalene.

As other additional active compounds for promoting the growth and/or limiting the loss of keratin fibers such as the hair and the eyelashes, that may be administered in combination with the compound of formula (I), which may or may not be salified and may or may not be solvated, exemplary are aminexil, 6-0-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharidic acids, substituted arylethylenes, N-acylamino acids, flavonoids, ascomycin derivatives and analogues, histamine antagonists, saponins, proteoglycanase inhibitors, oestrogen agonists and antagonists, pseudoterines, cytokines, growth factor promoters, IL-1 or IL-6 inhibitors, IL-10 promoters, TNF inhibitors, benzophenones, hydantoin, retinoic acid; vitamins, for instance vitamin D, vitamin B12 analogues and pantothenol; triterpenes, for instance ursolic acid and the compounds described in U.S. Pat. Nos. 5,529,769, 5,468,888 and 5,631,282; anti-pruriginous agents, for instance thenaldine, trimeprazine or cyproheptadine; anti-parasitic agents, in particular metronidazole, crotamiton or pyrethrinoids; calcium antagonists, for instance cinnarizine, diltiazem, nimodipine, verapamil, alverine and nifedipine; hormones such as oestriol or its analogues, thyroxine and its salts, and progesterone; FP receptor (type-F prostaglandin receptor) agonists such as latanoprost, (5E)-7-{(1R,2R,3R,5S)-3,5-dihydroxy-2-[(3R)-3-hydroxy-5-phenylpentyl]cyclopentyl}hept-5-enoic acid, bimatoprost, travoprost, unoprostone and butaprost; finasteride, triterpenes and potassium agonists; mixtures thereof.

Advantageously, the compositions according to the invention comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2, 17-phenyl PGE2,16,16-dimethyl PGF2-α, 17-phenyl PGF2-α, prostaglandins of series 1, for instance 11-deoxyprostaglandin E1, 1-deoxyprostaglandin E1 in salt or ester form, analogues thereof, especially latanoprost, (5E)-7-{(1R,2R,3R,5S)-3,5-dihydroxy-2-[(3R)-3-hydroxy-5-phenylpentyl]cyclopentyl}hept-5-enoic acid, viprostol, bimatoprost, cloprostenol, travoprost, fluprostenol, cloprostenol, butaprost, unoprostone, misoprostol, and the salts or esters thereof.

The composition preferably contains at least one non-prostanoic EP2 and/or EP4 receptor agonist as described especially in EP-1-175,892.

The composition comprising at least the compound of formula (I), salified or non-salified, solvated or non-solvated, may be in liposomal form, as described especially in WO 94/22468. Thus, the compound encapsulated in the liposomes may be delivered selectively to the hair follicle.

The compositions according to the invention may be applied to the alopecic areas of the scalp and the hair of an individual, and optionally left in contact for several hours and optionally rinsed off.

The compositions containing an effective amount of a compound of formula (I), salified or non-salified, solvated or non-solvated, may, for example, be applied in the evening, kept in contact throughout the night and optionally shampooed out in the morning. These applications may be repeated daily for one or more months according to the individual.

Advantageously, in the regime or regimen according to the invention, from 5 μL and 500 μL of a solution or composition as defined above, comprising from 0.001% to 5% of 15-PGDH inhibitor, are applied to the areas of the scalp to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Synthesis of the Compound (5Z)-5-[2-(4-tert-butylphenoxy)benzylidene]-1,3-thiazolidine-2,4-dione of Formula (I) (A₁=A₂=H; A₃=OZ₂; Z₂ is phenyl Substituted with a tert-butyl Group)

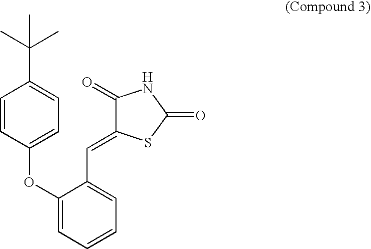

2-[4-(tert-Butyl)phenoxy]benzaldehyde (10 g; 39.3×10⁻³ mol) is diluted in 120 mL of toluene in a 250 mL round-bottomed flask on which is mounted Dean-Stark apparatus and a condenser, followed by addition of 2,4-thiazolidinedione (4.6 g; 39.9×10⁻³ mol), piperidine (1 mL) and acetic acid (1 mL). The reaction medium is refluxed for 4 hours. After cooling to room temperature, a precipitate forms, which is filtered off and then rinsed several times with toluene. The pale yellow solid obtained is dried under vacuum at 50° C. to give 8.54 g of product (yield: 62%).

### EXAMPLE 2

### Demonstration of the 15-PGDH-Specific Inhibitory Properties of the Compounds of Formula (I)

Test on Type-1 15-PGDH:

The enzyme 15-PGDH is obtained as described in patent application FR 02/05067 filed in the name of L'Oréal, as a suspension in a medium adjusted to a concentration of 0.3 mg/mL and then blocked at −80° C. For the purposes of the test, this suspension is thawed and stored in ice.

In parallel, a 100 mM, pH 7.4 Tris buffer containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), and 50 μM of Prostaglandin E₂ (P4172, Sigma-Aldrich, L'isle D'Abeau Chesne, BP 701, 38297, Saint Quentin Fallavier) is prepared.

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 mL of enzymatic suspension at 37° C. is introduced into the cuvette concomitantly with the recording (corresponding to an increase in the optical density at 340 nm). The maximum reaction rate is recorded.

The test values (containing the compounds of formula (I)) are compared with the control value (without compound of formula (I)); the results indicated represent either the percentage inhibition of the enzymatic activity of 15-PGDH for a given concentration of compound of formula (I), or the concentration at which the compound of formula (I) reduces the enzymatic activity of 15-PGDH by 50%, namely IC_{50dh}.

The results are reported in the table below.

| | | IC_{50dh} or % |
|---|---|---|
| Compound | Structure | inhibition |
| 1 | 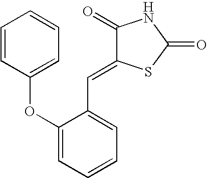 | 12 μM |
| 2 | 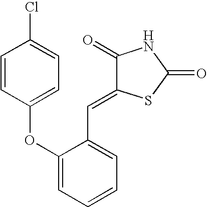 | 5 μM |
| 3 | 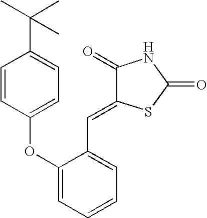 | 0.08 μM |
| 4 | 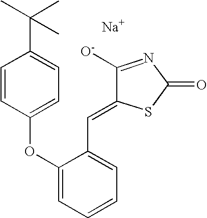 | 0.094 μM |
| 5 | 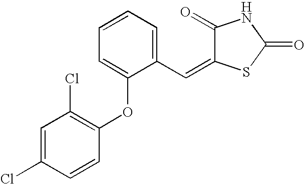 | 3 μM |
| 6 | 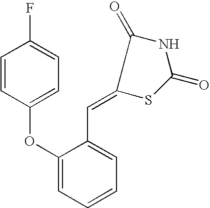 | 48% at 100 μM |
| 7 | 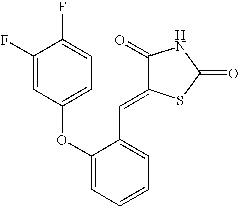 | 1.3 μM |
| 8 | 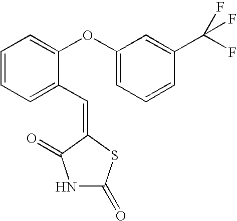 | 2.4 μM |
| 9 | 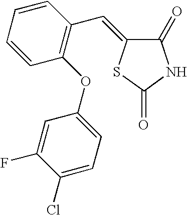 | 0.46 μM |
| 10 | 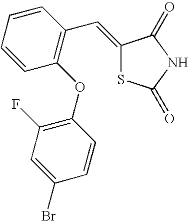 | 1.55 μM |
| 11 | 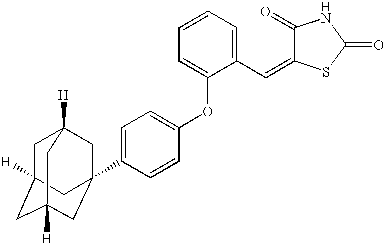 | 1.4 μM |
| 12 | 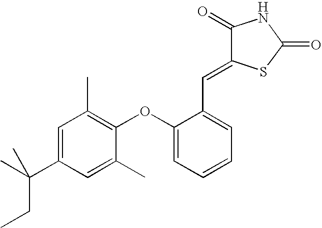 | 8 μM |
| 13 | 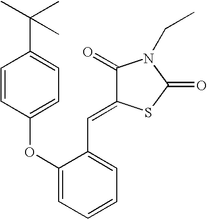 | 23% at 100 μM |
| 14 | 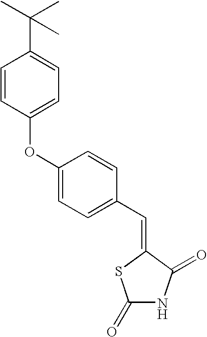 | 0.085 μM |
| 15 | 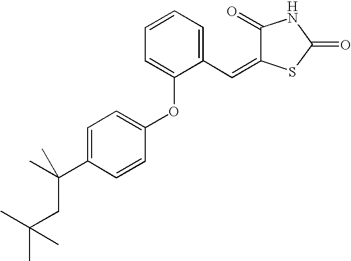 | 27% at 100 μM |
| 16 | 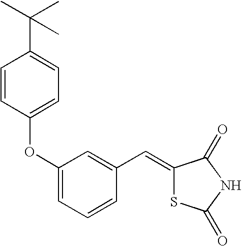 | 0.20 μM |
| 17 | 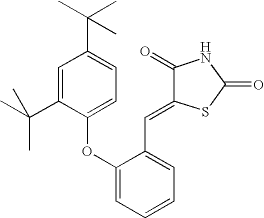 | 30% at 100 μM |

From this table it is clearly seen that the compounds of formula (I) are inhibitors of type-1 15-PGDH.

The compositions below are formulated via the usual techniques commonly employed in cosmetics or pharmaceutics.

### EXAMPLE 3

### Hair Lotion

| | |
|---|---|
| Compound (5Z)-5-[2-(4-tert-butylph- | 1.00 g |
| enoxy)benzylidene]-1,3-thiazolidine-2,4-dione | |
| Propylene glycol | 30.00 g |
| Ethyl alcohol | 40.00 g |
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to reduce the loss and promote regrowth of the hair. It also makes it possible to improve the appearance of the hair.

### EXAMPLE 4

### Hair Lotion

| | |
|---|---|
| Compound (5Z)-5-[2-(4-tert-butyl- | 1.00 g |
| phenoxy)benzylidene]-1,3-thiazolidine-2,4-dione | |
| Ethyl alcohol | 49.00 g |
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to reduce the loss and promote regrowth of the hair. It also makes it possible to improve the appearance of the hair.

### EXAMPLE 5

### Wax/Water Mascara

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound (5Z)-5-[2-(4-tert-butyl- | 1.00% |
| phenoxy)benzylidene]-1,3-thiazolidine-2,4-dione | |
| Black pigment | 5.00% |
| Preservative | qs |
| Water | qs 100.00% |

This mascara is applied to the eyelashes like a standard mascara with a mascara brush. It makes it possible to reduce the loss and to promote regrowth of the eyelashes. This mascara also makes it possible to improve the appearance of the eyelashes.

### EXAMPLE 6

### Hair Lotion

| | |
|---|---|
| Compound (5Z)-5-[2-(4-tert-butyl- | 0.10 g |
| phenoxy)benzylidene]-1,3-thiazolidine-2,4-dione | |
| Latanoprost | 0.10 g |
| Propylene glycol | 30.00 g |
| Ethyl alcohol | 40.00 g |
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to reduce the loss and promote regrowth of the hair. It also makes it possible to improve the appearance of the hair.

### EXAMPLE 7

### Hair Lotion

| | |
|---|---|
| (5E)-7-{(1R,2R,3R,5S)-3,5-Dihydroxy-2-[(3R)-3- | 0.10 g |
| hydroxy-5-phenylpentyl]cyclo-pentyl}hept-5-enoic | |
| acid | |
| Compound (5Z)-5-[2-(4-tert-butyl- | 0.10 g |
| phenoxy)benzylidene]-1,3-thiazolidine-2,4-dione | |
| Propylene glycol | 30.00 g |
| Ethyl alcohol | 40.00 g |
| Water | qs 100.00 g |

This lotion is applied to the scalp, once or twice a day, at a rate of 1 ml per application, while massaging the scalp gently to make the active agent penetrate. The head of hair is then dried in open air. This lotion makes it possible to reduce the loss and promote regrowth of the hair. It also makes it possible to improve the appearance of the hair.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for inducing and/or stimulating the growth of mammalian keratin fibers and/or reducing the loss and/or increasing the density thereof, comprising administering to a subject in need of such treatment, for such period of time as required to elicit the desired effect, a thus effective amount of at least one benzylidene-1,3-thiazolidine-2,4-dione compound having the following structural formula (I): 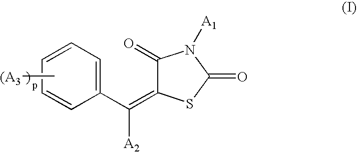
in which:
a) A₁ and A₂ simultaneously are hydrogen;
b) A₃ is a group Z₁;
c) Z₁ is a group OZ₂ in which Z₂ is a phenyl group optionally substituted with one or more adamantyl, CF₃, halogen or linear or branched, saturated or unsaturated C₁-C₂₀ alkyl radicals; and
d) p ranges from 0 to 5 inclusive and, when p>1, the substituents A₃ may be identical or different; or salt and/or solvate and/or cis-trans or Z/E isomer thereof.

2. A regime or regimen for inhibiting 15-hydroxyprostaglandin dehydrogenase in a mammal in need of such treatment, comprising administering thereto a thus effective amount of at least one benzylidene-1,3-thiazolidine-2,4-dione compound as defined in claim 1, or salt and/or solvate and/or isomer thereof.

3. A regime or regimen for inhibiting human skin type-1 15-hydroxyprostaglandin dehydrogenase in an individual in need of such treatment, comprising administering thereto a thus effective amount of at least one benzylidene-1,3-thiazolidine-2,4-dione compound as defined in claim 1, or salt and/or solvate and/or isomer thereof.

4. The regime or regimen as defined by claim 1, wherein said at least one benzylidene-1,3-thiazolidine-2,4-dione compound of formula (I) is selected from the following list of compounds, or salt and/or solvate and/or isomer thereof:

| Com- | |
|---|---|
| pound | Structure |
| 1 | 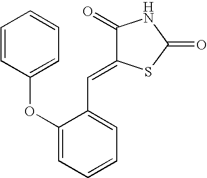 |
| 2 | 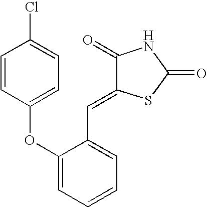 |
| 3 | 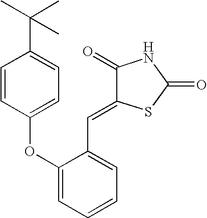 |
| 4 | 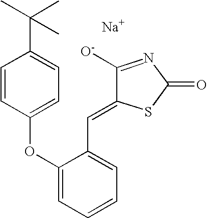 |
| 5 | 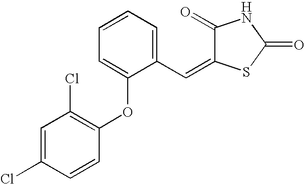 |
| 6 | 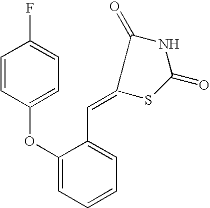 |
| 7 | 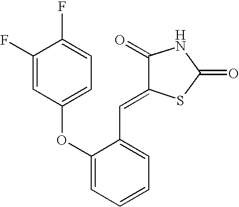 |
| 8 | 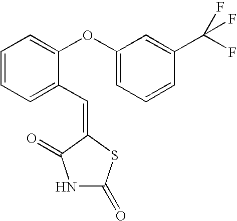 |
| 9 | 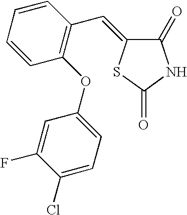 |
| 10 | 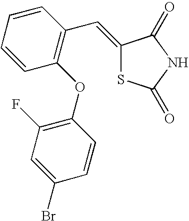 |
| 11 | 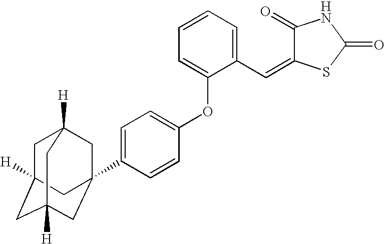 |
| 12 | 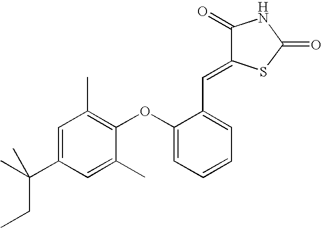 |
| 13 | 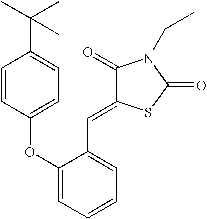 |
| 14 | 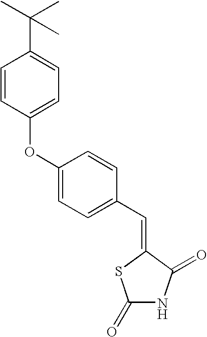 |
| 15 | 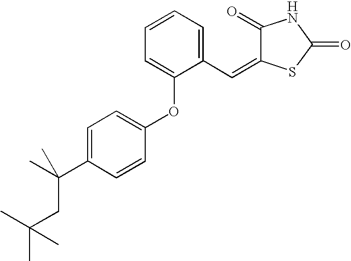 |
| 16 | 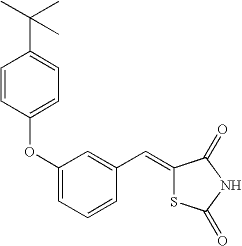 |
| 17 | 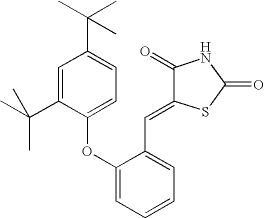 |

5. A benzylidene-1,3-thiazolidine-2,4-dione compound having the following structural formula (Ia): 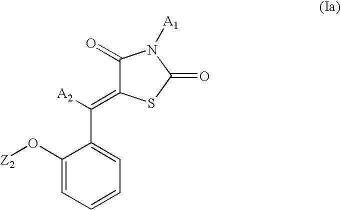
in which:
A₁ and A₂ are hydrogen; and
Z₂ is a phenyl group substituted with one or more adamantyl or linear or branched, saturated or unsaturated C₂-C₂₀ alkyl radical; other than tert-butyl and isobutyl, or salt and/or solvate and/or isomer thereof.

6. The compound (5Z)-5-[2-[4-(1-adamantylphenoxy)benzylidene]-1,3-thiazolidine-2,4-dione of structure: 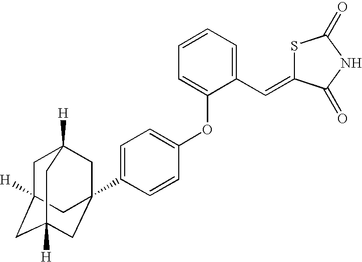
or salt and/or solvate and/or isomer thereof.

7. The compound (5Z)-5-[2-(2,6-dimethyl-4-tert-pentylphenoxy)benzylidene]-1,3-thiazolidine-2,4-dione of structure: 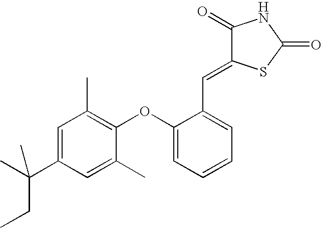
or salt and/or solvate and/or isomer thereof.

8. The compound of structure: 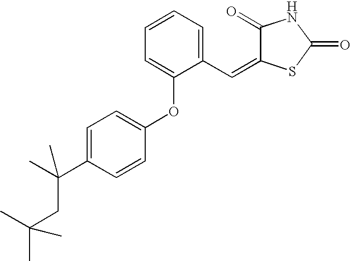
or salt and/or solvate and/or isomer thereof.

9. A regime or regimen for inducing and/or stimulating the growth of human head hair, beard hair, moustache hair, eyelashes and/or eyebrows of an individual in need of such treatment, comprising topically applying thereon a thus effective amount of at least one benzylidene-1,3-thiazolidine-2,4-dione compound as defined in claim 1, or salt and/or solvate and/or isomer thereof.

10. A regime or regimen for reducing the loss and/or increasing the density of human head hair, beard hair, moustache hair, eyelashes and/or eyebrows of an individual in need of such treatment, comprising topically applying thereon a thus effective amount of at least one benzylidene-1,3-thiazolidine-2,4-dione compound as defined in claim 1, or salt and/or solvate and/or isomer thereof.

11. A regime or regimen for treating ando-chrono-genetic alopecia and/or for treating alopecia of natural origin, comprising administering to an individual in need of such treatment, for such period of time as required to elicit the desired effect, a thus effective amount of at least one benzylidene-1,3-thiazolidine-2,4-dione compound as defined in claim 1, or salt and/or solvate and/or isomer thereof.

12. A regime or regimen for treating a disorder associated with human skin tupe-1 15-hydroxyprostaglandin dehydrogenase, comprising administering to an individual in need of such treatment, for such period of time as required to elicit the desired effect, a thus effective amount of at least one benzylidene-1,3-thiazolidine-2,4-dione compound as defined in claim 1, or salt and/or solvate and/or isomer thereof.

13. A regime or regimen for caring for and/or making up human keratin fibers, comprising topically applying thereon a thus effective amount of at least one benzylidene-1,3-thiazolidine-2,4-dione compound as defined in claim 1, or salt and/or solvate and/or isomer thereof.

14. A cosmetic/therapeutic composition comprising an amount of at least one benzylidene-1,3-thiazolidine-2,4-dione compound as defined in claim 1, effective to induce and/or to stimulate the growth and/or to reduce the loss and/or increase the density of keratin fibers, formulated into a non-toxic physiologically acceptable medium therefor.

15. The cosmetic/therapeutic composition as defined by claim 14, formulated for topical application.

16. The cosmetic/therapeutic composition as defined by claim 14, comprising from 10⁻³ to 10% of said at least one benzylidene-1,3-thiazolidine-2,4-dione compound.

17. The cosmetic/therapeutic composition as defined by claim 14, further comprising at least one prostaglandin and/or derivative thereof.

18. The cosmetic/therapeutic composition as defined by claim 14, formulated as a mascara.

19. A cosmetic process for treating human keratin fibers and/or the skin from which said fibers emerge, to improve their condition and/or appearance, comprising topically applying to the keratin fibers and/or the skin a cosmetic composition comprising at least one compound of formula (I) as defined in claim 1 or salt and/or solvate and/or isomer thereof, maintaining same in contact with the keratin fibers and/or the skin from which the said fibers emerge, and optionally rinsing the said fibers and/or the said skin.
